(19) United States
(12) Patent Application Publication
(10) Pub. No.: US 2015/0351957 A1
(43) Pub. Date: Dec. 10, 2015

(54) PREVENTION & TREATMENT OF NEUROPATHY

(71) Applicant: NATIONAL UNIVERSITY OF SINGAPORE, Singapore (SG)

(72) Inventors: Einar Wilder-Smith, Singapore (SG); Kaiquan Shen, Singapore (SG)

(73) Assignee: National University of Singapore, Singapore (SG)

(21) Appl. No.: 14/761,239

(22) PCT Filed: Jan. 27, 2014
(86) PCT No.: PCT/SG2014/000031

§ 371 Date: Jul. 15, 2015

(30) Foreign Application Priority Data
Feb. 1, 2013 (GB) 1301816.3

(51) Int. Cl. A61F 7/10; A61B 5/01; A61B 5/026

(52) U.S. Cl. CPC A61F 7/10; A61B 5/026; A61B 5/01; A61F 2007/0056

(57) ABSTRACT

The invention relates to the use of hypothermia to prevent or treat peripheral nerve neuropathy and including a system to induce optimized cooling of one or more body parts, typically the limbs.

### FIELD OF THE INVENTION

The invention relates to the use of hypothermia to prevent or treat peripheral nerve neuropathy and includes a system to induce optimized cooling of one or more body parts, typically the limbs.

### BACKGROUND OF THE INVENTION

Peripheral neuropathy is a condition that affects the peripheral nerves and can be caused by a number of conditions and treatments of conditions such as cancer, HIV infection, Herpes simplex infection, leprosy and diseases such as rheumatoid arthritis and automimmune diseases that affect the peripheral nervous system such as Guillian-Barr Syndrome. The symptoms of peripheral neuropathy include weakness, loss of sensation, pain, bladder and sexual dysfunction, abnormal blood pressure and heart function. In some examples the cause of peripheral neuropathy is not apparent and is referred to as idiopathic. A common causative agent of peripheral neuropathy is chemotherapy in the treatment of cancer which is referred to as chemotherapy induced neuropathy [CIN].

Chemotherapy is the treatment of cancer with one or more cytotoxic drugs causing the death of healthy and malignant cells. The inability of chemotherapeutic agents to differentiate between cancerous and healthy cells results in a variety of dose-dependent side effects. Common experienced side effects are nausea, hair loss, fatigue or pain. Chemotherapy induced neuropathy is another severe side effect which occurs in a high majority of cancer patients after treatment with chemotherapeutics. Symptoms of peripheral neuropathy are usually mild to begin with and gradually worsen affecting frequently the hands, feet and lower legs. Symptoms include a change in sensation, increased sensitivity, mild to severe pain, numbness, muscle weakness and dizziness.

Several chemotherapeutic agents are known to cause peripheral neuropathy as for example vincristine and vinca alkaloids, platinum compounds e.g. cisplatin, oxaliplatin, carboplatin, taxanes, epothilones, bortezomib (a first line agent in multiple myeloma) and thalidomide; however, the mode of action of these drugs causing the nerve damage is commonly unknown. Platinum compounds, as for example, oxaloplatin are thought to accumulate in the dorsal root ganglia and produce hyperexcitability, whereas vinca alkaloids induce alterations in the cellular micro-tubuli structure leading to disruption of the axonal flow. The neurotoxicity of these agents is type and dose dependent, and severity of neuropathy is increasing with duration of treatment. CIN can occur in up to 90% of patients and can have irreversible effects. Paclitaxel was found to induce CIN in 57%-83%, vinca alkaloids induced CIN in 30% to 47%, cisplatin ranges from 28% to 100% and ixabepilone was found to induce CIN in around 67% of patients.

Currently there are no effective treatment methods available to prevent or cure CIN. Some neuroprotective agents are thought to decrease the neurotoxicity of the chemotherapeutic agent; however, there is no concrete clinical evidence supporting this data and the compounds to prevent CIN are known to cause side effects as well. Omega-3 fatty acids are thought to have neuroprotective function in Paclitaxel-induced peripheral neuropathy in breast cancer patients; however, these results have to be confirmed.

As there are no curative treatment options known for CIN sufferers and the use of chemotherapeutics is unavoidable, novel ways to prevent nerve damage after treatment with these drugs is prerequisite. This will ease the financial burden on the health care system and allow patients after chemotherapy leading a more normal life.

Hypothermia has been used to provide neuroprotection and improve the neurological outcomes after brain ischemia. Several clinical trials have shown hypothermia as being effective in reducing central nervous system neuronal damage in patients after cardiac arrest. Although the ultimate mechanism for hypothermic neuroprotection remains unknown, there is some emerging evidence that hypothermia provides neuroprotection through inhibition of a variety of cellular metabolic processes of which reduction of AMP-Activated Protein Kinases has been recently implicated as a possible key factor. In small animal studies, cooling prevents neuronal (central nervous system cells) death as well as inflammation and many associated detrimental neurochemical changes induced by noxious stimuli such as ischemia, trauma and toxic substances.

The effects of hypothermia on peripheral nerve damage are less studied. A recent murine study investigated the effect of hypothermia on peripheral nerves damaged by crush trauma. The study showed a beneficial effect of hypothermia on preventing peripheral nerve pain induced by sciatic nerve crush. Hypothermia was also found to reduce the effects of nerve anoxia, and interestingly, hypothermia during anoxia allows better recovery of nerve functions than constant hypothermia. This seems to indicate that protective mechanisms may be particularly effective when applied during the actual time of nerve damage. Furthermore the extent of protective hypothermia seems to have different effects on nerve function preservation as determined by nerve, function studies using nerve conduction. The nerve action potential amplitude, an indicator of the number of functioning axons within a nerve, was optimally preserved in nerves rendered hypoxic, when the nerve was cooled at 17° C. Nerve conduction velocity, an indicator of nerve myelination, was best preserved at temperatures around 21° C.

Hypothermia treatment is also thought to avert Chemotherapy Induced Alopecia (CIA) or thinning of the hair, a frequent occurring side effect during chemotherapy. Patients are offered to wear a “cold cap” during treatment preventing hair loss. CIA is thought to be a result of toxic accumulation of chemotherapeutics in the hair follicle. The protective effect of scalp cooling is thought to be due to vasoconstriction of the skin vessels, resulting in lower doses of toxic substances reaching the hair follicles as well as reduced biochemical activity in the hair follicles.

The lowering of the patient's body temperature can be achieved by chilled blankets, torso vest and leg wraps in direct contact with the patient's skin. WO2012/162199 discloses a portable apparatus for the immersion of hands and forearms in cooling water for the reduction of core temperature of human beings experience exertion heat stroke. US2012/0310312 discloses a head cooling system inducing “the diving reflex” which results in a reduced heart rate, metabolism and preferred transport of oxygen to the heart and brain preserving the viability of these organs.

The applicant surprisingly found that cooling of the arms and legs results in a decrease of nerve function. The decreased temperature primarily reduces the blood flow supplied to the peripheral nerves and toxic chemotherapeutics are directed away from the cooled organ/tissue and hence reduces neurotoxicity. The disclosure utilizes hypothermia in a carefully designed way to prevent CIN. Since the nerves damaged in CIN are peripheral, a device is deployed to the arms and legs, which senses, regulates and maintains optimal conditions to prevent CIN. Important constituents for this biofeedback system may include nerve and skin tissue blood flow, oxygenation and temperature monitoring.

### STATEMENTS OF INVENTION

According to an aspect of the invention there is provided a device for inducing regulated controlled hypothermia in a subject comprising: a body part covering comprising flexible material adapted to contact the skin of a subject and having an upper and lower surface defining a space for coolant, the device provided with one or more inlets for fluid coolant connecting to a closed network of channels to facilitate even distribution of fluid within the device space and describing a coolant dispersal pattern when in use to provide uniform cooling to the subjects body part and wherein the device is further provided optionally with one or more temperature sensors and optionally one or more blood flow sensors to monitor the subjects body part temperature and/or the blood flow through the subjects body part.

In a preferred embodiment of the invention the device is adapted to cover at least partially the upper limb[s] of the subject.

In a preferred embodiment of the invention said device is adapted to cover at least the hands and forearm of the subject. In an alternative preferred embodiment of the invention said device is adapted to cover at least partially the lower limb[s] of the subject.

In a preferred embodiment of the invention said device is provided with at least one temperature sensor and at least one blood flow sensor in functional contact with the hand, forearm and/or lower limb of the subject.

In a preferred embodiment of the invention said device is provided with a plurality of temperature sensors and a plurality of blood flow sensors in functional contact with the hand, forearm and/or lower limb of the subject.

In a preferred embodiment of the invention said device is provided with multiple inlets.

In preferred embodiment of the invention the channels are formed from a plurality of upwardly projecting fins which are positioned near to each other within the cavity of the body part covering to create a network of channels throughout the body part covering.

In a preferred embodiment of the invention the fins are positioned equidistant from each other within the cavity of the body part covering.

In a preferred embodiment of the invention said body covering is provided with more than one sensor functionally linked in series to provide continuous temperature and/or blood flow monitoring of said subject over all or part of the subjects body part and adjusting the cooling means to control the induced hypothermia.

In a preferred embodiment of the invention said blood flow sensor[s] detect the concentration of haemoglobin.

In an alternative preferred embodiment of the invention said blood flow sensor[s] detect the oxygenation of haemoglobin.

In a further preferred embodiment of the invention said device further comprises a plurality of micro temperature probes adapted to measure the skin temperature of the subject's body part when in use and wherein the micro temperature probes are positioned to provide a measure of the skin temperature over the area of the patient's body contacted by the device.

In an alternative preferred embodiment of the invention said flexible material is manufactured from a material transparent to light or optically clear.

Preferably said material is selected from the group: polydimethylsiloxane (PDMS), indium tin oxide (ITO) and flexible ultra-thin glass film.

According to an aspect of the invention there is provided an apparatus for inducing and regulating controlled hypothermia in a subject to prevent or treat peripheral nerve neuropathy wherein said apparatus comprising:
a device according to the invention wherein the device is functionally connected to a temperature and/or blood flow monitor to monitor temperature and/or blood flow in the subjects body part via the temperature and/or blood flow sensors;
cooling means which when is use delivers coolant to the body covering which induces hypothermia in the subject and is regulated when in use to provide optimal cooling to the subject.

In a preferred embodiment of the invention said apparatus includes a photoacoustic microscopy system for the monitoring of blood flow.

According to a further aspect of the invention there is provided a method to monitor nerve temperature during chemotherapy induced hypothermia in a subject comprising:
i) providing a device according to the invention wherein the device includes a plurality of micro temperature probes and connecting the device to said subject; and
ii) monitoring the skin temperature of the subject during induced hypothermia.

According to a further aspect of the invention there is provided a method for the continuous monitoring of blood flow and/or blood oxygenation through a subject's body part during induced hypothermia and chemotherapy comprising:
i) providing an device according to the invention wherein the device is manufactured from transparent material and applying the device to a subject in need of chemotherapy;
ii) exposing the device to a light source; and
iii) measuring the flow of blood and/or oxygenation through and of the body part.

In a preferred method of the invention the wavelength of light is 620-750 nm.

In an alternative method of the invention the wavelength of light is 850 nm-1 mm.

In a further preferred method of the invention the light source alternates between 620-750 nm and 850 nm-1 mm.

In a preferred method of the invention the concentration of haemoglobin is monitored as a measure of blood flow.

In an alternative preferred method of the invention the oxygenation of haemoglobin is monitored as a measure of blood flow.

In a preferred method of the invention blood flow is monitored using confocal functional photoacoustic microscopy.

In a preferred method of the invention the wavelength of light during photoacoustic microscopy is between 650-500 nm.

In a further preferred method of the invention the wavelength of light during photoacoustic microscopy is 560 and 570 nm.

According to a further aspect of the invention there is provided a method to prevent or treat peripheral nerve neuropathy comprising:
i) providing an device according to the invention and connecting the device to a subject;
ii) cooling the subject to induce hypothermia in one or more parts of the subjects body;
iii) monitoring and controlling the temperature of the subject to maintain induced hypothermia.

In a preferred method of the invention the temperature of, the subject's body part is reduced to between 15° C. and 25° C. Preferably, the temperature of the subject's body part is reduced to around 20° C.

In a preferred method of the invention the temperature of the subject's body is reduced to between 23° C. and 25° C.

In a preferred method of the invention induced hypothermia is induced for a period, for example 30 minutes before administration of chemotherapy. Preferably induced hypothermia is maintained throughout the administration of chemotherapy.

In a preferred method of the invention the induced hypothermia is, maintained for a period (preferably 30 minutes) after administration of chemotherapy.

In a preferred method of the invention the induced hypothermia is maintained for a period of 3 hours after administration of chemotherapy; preferably the temperature of the subject's body is reduced to between 23° C. and 25° C.

In a preferred method of the invention said subject is human.

In a preferred method of the invention the peripheral nerve neuropathy is chemotherapy induced neuropathy as a result of cancer treatment.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “cancer” includes malignancies of the various organ systems, such as those affecting, for example, lung, breast, thyroid, lymphoid, gastrointestinal, and genito-urinary tract, as well as adenocarcinomas which include malignancies such as most colon cancers, renal-cell carcinoma, prostate cancer and/or testicular tumours, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term “carcinoma” also includes carcinosarcomas, e.g., which include malignant tumours composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

A general definition of “chemotherapy” is the use of an agent that typically is a small chemical compound that kills cells in particular diseased cells or is at least cytostatic. Agents can be divided with respect to their structure or mode of action. For example, chemotherapeutic agents include alkylating agents, anti-metabolites, anthracyclines, alkaloids, plant terpenoids and topoisomerase inhibitors. Chemotherapeutic agents typically produce their effects on cell division or DNA synthesis. Examples of alkylating agents are is cisplatin, carboplatin or oxaliplatin. Examples of anti-metabolites include purine or pyrimidine analogues. Purine analogues are known in the art. For example thioguanine is used to treat acute leukaemia. Fludarabine inhibits the function of DNA polymerases, DNA primases and DNA ligases and is specific for cell-cycle S-phase. Pentostatin and cladribine are adenosine analogues and are effective against hairy cell leukaemias. A further example is mercaptopurine which is an adenine analogue. Pyrimidine analogues are similarly known in the art. For example, 5-fluorouracil (5-FU), floxuridine and cytosine arabinoside. 5-FU has been used for many years in the treatment of breast, colorectal cancer, pancreatic and other cancers. 5-FU can also been formed from the pro-drug capecitabine which is converted to 5-FU in the tumour. Alkylating agents are also known in the art and include vinca alkaloids, for example vincristine or vinblastine. Terpenoids have been used for many years and include the taxanes, for example, paclitaxel.

In a preferred method of the invention the chemotherapy induced neuropathy is the result of administration of a chemotherapeutic agent selected from the group consisting of: vincristine, vinca alkaloids, platinum compounds such as cisplatin, oxaliplatin, carboplatin, taxanes, epothilones, bortezomib and thalidomide.

In an alternative preferred method of the invention peripheral nerve neuropathy is infection induced. Preferably said infection is an HIV infection.

Peripheral neuropathy as a consequence of HIV infection can be due either to direct viral infection or as a consequence of administration of anti-viral chemotherapy using, for example, nucleoside reverse transcriptase inhibitors such as didanosine, zalcitabine or stavadine.

In a further alternative method of the invention peripheral nerve neuropathy is associated with an inflammatory neural disease.

There is a vast array of diseases exhibiting a chronic inflammatory component many of which are chronic neural inflammatory diseases for example, chronic inflammatory demyelinating polyradiculoneuropathy, chronic inflammatory demyelinating polyneuropathy, multiple sclerosis, Guillan-Barre Syndrome, Friedreich's ataxia, systemic lupus erythema and myasthemia gravis. It is apparent that many diseases have an inflammatory component many of which are autoimmune diseases.

In a preferred method of the invention said disease is Guillian-Barre Syndrome.

In a still further alternative method of the invention peripheral nerve neuropathy is associated with an endocrine or metabolic disease.

In a preferred method of the invention said endocrine or metabolic disease is selected from the group consisting of: diabetes mellitus, kidney disease, porphyria, liver disease or hypothyroidism.

In a preferred method of the invention chemotherapy treatment is administered before, during or after induction of hypothermia.

In a preferred method of the invention said subject is precooled prior to administration of a chemotherapeutic agent.

In a preferred method of the invention induced hypothermia is combined with the administration of neuroprotective agents.

According to a further aspect of the invention there is provided the use of the device according to the invention in the prevention and treatment of chemotherapy induced neuropathy.

According to a further aspect of the invention there is provided the use of the apparatus according to the invention in the prevention and treatment of chemotherapy induced neuropathy.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

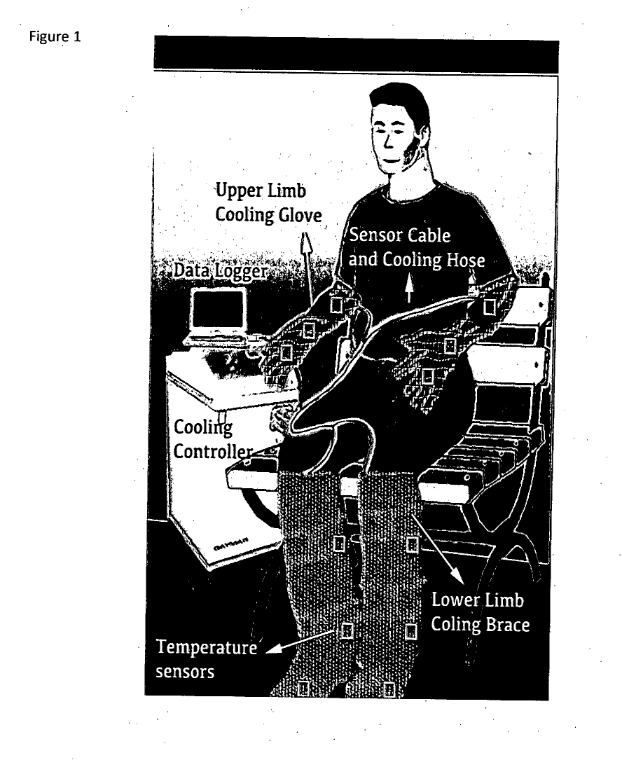

FIG. 1: is an artist impression of the proposed device for PREvention of CHemotherapy Induced Neuropathy (PRECHIN). It can receive feedback signals via the temperature sensors to control the temperature of coolant for maximal peripheral nerve protection during chemotherapy;

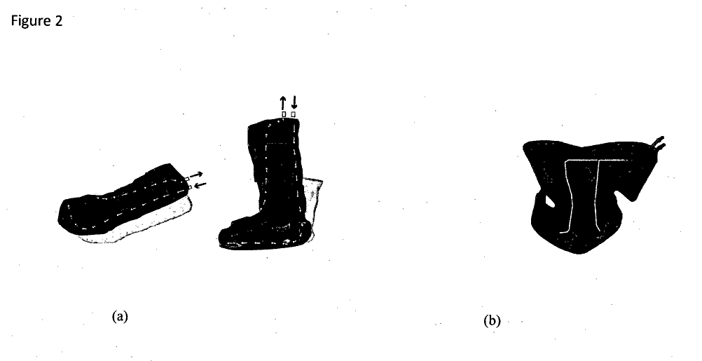

FIG. 2: illustrates arm and leg cooling wraps (a) elastic arm and leg cooling wrap design for tight and direct contact to patient's skin, (b) the coolant dispersal pattern to allow fluid coolant to continuously perfuse the device when in use to provide uniform cooling to the subjects body part;

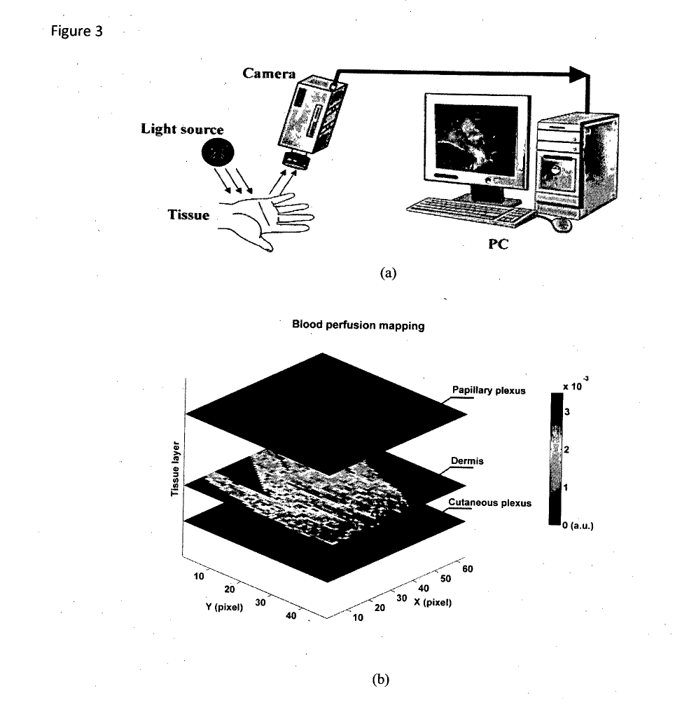

FIG. 3: illustrates remote iPPG (Photoplethysmography): (a) the setup of remote iPPG for measure perfusion changes due to temperature changes, (b) sample figure of layered PPG imaging of blood perfusion in the palm. The color indicates the intensity (a.u.);

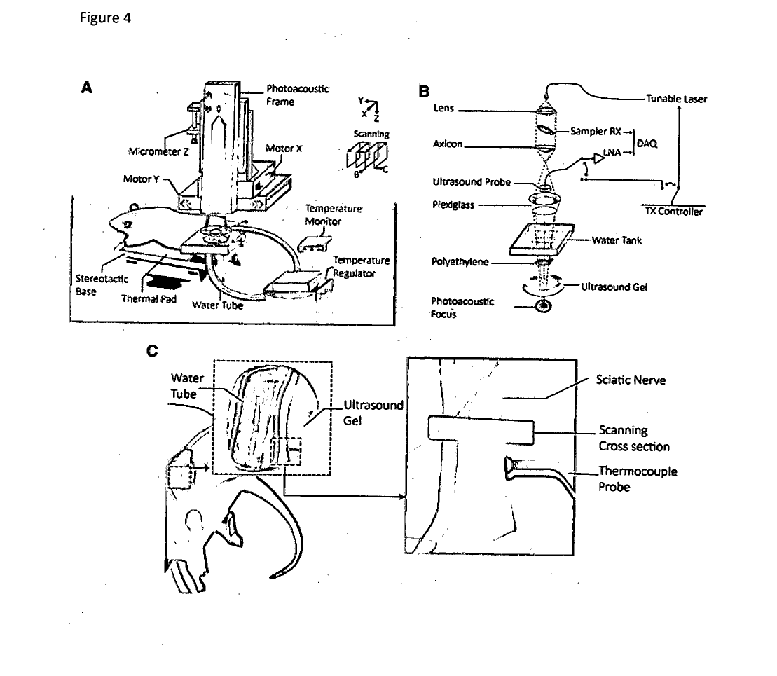

FIG. 4: (A) Experimental dark-field fPAM system integrated with the thermoregulation setup. Commercially available ultrasound gel was applied on the rat sciatic nerve for acoustic and thermal coupling; the rat subjects were placed between the water container and a custom-made stereotaxic apparatus for imaging. (B) The laser was pulsed with frequency of 10 Hz and coupled to an optical fibre into the strong focusing dark-field PA path to illuminate the target cross-section at the nerve. PA waves were detected by a 50-MHz transducer and then through the A/D card to the computer for further data analysis. (C) Localized temperature modulation was achieved via immediate heat transfer between the sciatic nerve and the perfused thermoregulatory water tube. A fixed scanning cross-section was selected during all experiments. The nerve thermocouple couple probe was placed directly below the sciatic trifurcation. We also applied sutures as needed to reinforce the stability of the tube and thermocouple probe;

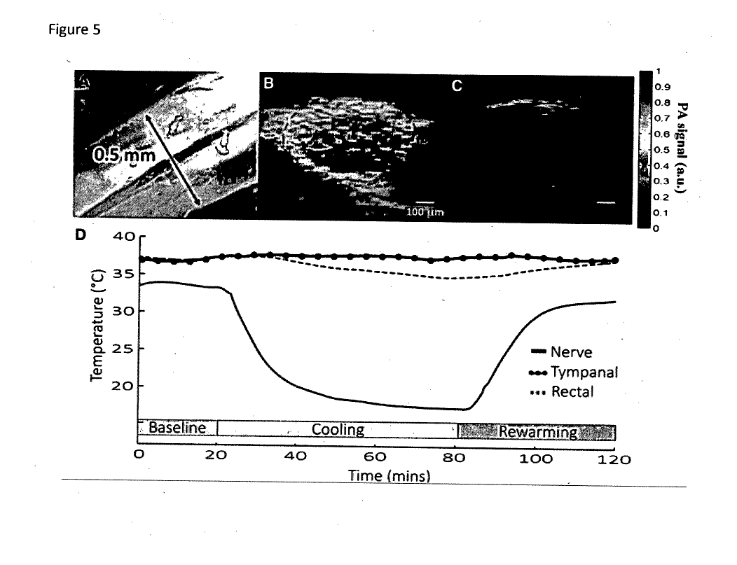

FIG. 5: (A) Photograph of the sciatic nerve (about 0.5 mm) showing several blood vessels from the epineurial vascular plexus. (B) Ultrasound and (C) PA cross-sectional B-scan images of the sciatic nerve. The yellow scale bars are equivalent to 50 μm. The ROI with the PA signal changes in scanned sciatic nerve image section was identified by the ultrasound image, as indicated by the red dashed line in (B). (D) Localized nerve thermoregulation protocol, including the baseline, cooling and rewarming. Temperature changes in the sciatic nerve, tympanic and rectal areas were monitored.

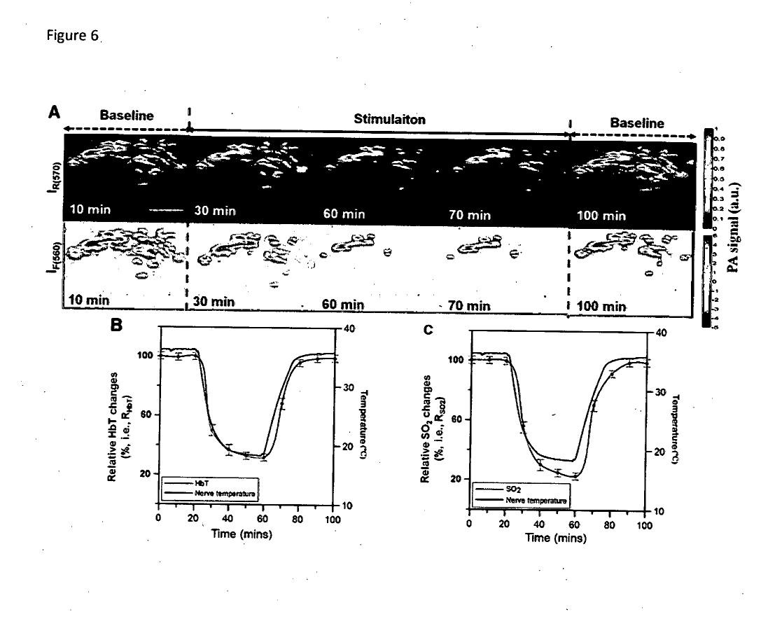

FIG. 6: (A) In vivo relative I(570) (i.e., HbT; upper panel) and I_F(560) (i.e., SO₂; lower panel) PA B-scan images of selected position at different times of temperature modulation protocol. Note that the I(570) and I_F(560) are specifically sensitive to relative HbT and SO₂ changes, respectively. The red scale bar is equivalent to 50 μm and applies to all images in panel A. Mean functional (B) HbT (i.e., R_{HbT}) and (C) SO₂ (i.e., R_{SO}_{₂}_{—}2) changes resulting from in vivo temperature modulation of the rat sciatic nerve as a function of time. The error bars indicate standard deviation (n=10).

### MATERIALS & METHODS

### Dark-Field Confocal Functional Photoacoustic Microscopy System

Our 50-MHz dark-field confocal fPAM system for imaging functional hemodynamics in the sciatic nerve is shown in FIG. 4 consisting of laser pulse generation and delivery (FIG. 4A), PA signal reception, and image reconstruction and display (FIG. 4B). Laser pulses, 4 ns wide, were generated at a frequency of 10 Hz by using an optical parametric oscillator (Surlite OPO Plus, Continuum, USA). The laser was pumped by a frequency-tripled Nd:YAG Q-switched laser (Surlite II-10, Continuum, USA). Two visible wavelengths of the laser pulses, 560 and 570 nm (λ₅₆₀ and λ₅₇₀), were employed for PA wave excitation [51]. At the selected wavelengths, blood is a dominant optical absorber, producing strong optical absorption and thus guaranteeing that the detected PA signals mainly come from blood [50,51]. The acquired PA signal at λ₅₆₀ is sensitive to relative changes in SO₂, while relative HbT changes are the most prominent at λ₅₇₀ [50]. The 50-MHz ultrasonic transducer used in the current fPAM system was custom-made by the Acoustic Sensor Co., Ltd at Taiwan. It has a −6 dB fractional bandwidth of 57.5%, a focal length of 9 mm and a 6 mm active element, offering an axial resolution of 32 μm and a lateral resolution of 61 μm.

Laser energy was delivered using a 1-mm multimodal fiber (Thorlabs, U.S.A). The fiber tip was coaxially aligned with a convex lens, an axicon, a plexiglass mirror, and an ultrasonic transducer on an optical bench, forming dark-field illumination that was confocal with the focal point of the ultrasonic transducer. The incident energy density on the sample surface was well within American National Standards institute (ANSI) safety limits. The transducer was immersed in an acrylic water tank during the imaging process, and the hole at the bottom of the tank was sealed with a piece of 15-μm thick polyethylene film. A thin layer of ultrasonic gel was applied as a PA and thermal conductive medium, which was then attached to the thin polyethylene film to ensure reliable coupling of the PA waves with the water tank. The PA signals received by the ultrasonic transducer were pre-amplified by a low-noise amplifier (noise figure 1.2 dB, gain 55 dB, AU-3A-0110, USA), cascaded to an ultrasonic receiver (5073 PR, Olympus, USA) and then digitized and sampled by a computer-based 14-bit analog to digital (A/D) card (CompuScope 14220, GaGe, USA) at a 200-MHz sampling rate for data storage.

Fluctuations in the laser energy were monitored with a photodiode (DET36A/M, Thorlabs, USA). The recorded photodiode signals were measured prior to the experiment to compensate for PA signal variations caused by laser-energy instability. The achievable penetration depth of the current fPAM system was 3 mm with approximately 18-dB SNR, where SNR is defined as the ratio of the signal peak value to the root-mean-square value of the noise. Three scan types can be provided by this system (FIG. 4A): A-line (i.e., one-dimensional images where the axis represents the imaging depth), B-scan (i.e., two-dimensional images where one axis is the lateral scanning distance and the other is the imaging depth), and C-scan (i.e., projection images from the three-dimensional images) [56]. The amplitude of the envelope-detected PA signals was used in the subsequent functional imaging analysis [50].

### EXPERIMENTAL ANIMALS

Ten adult female Wistar rats (NUS-CARE, Singapore) weighing 280±20 grams were used and housed at a constant temperature and humidity with free access to food and water. The Institutional Animal Care and Use Committee (IACUC) at the National University of Singapore approved all the experimental procedures.

Rats remained anesthetized with isoflurane 2-3% in 100% O₂ and were mounted on a dorsal position over a custom-made acrylic stereotaxic holder. Next, the left hind limb was shaved and disinfected prior to making a 40 mm longitudinal incision at knee level. The biceps femoris was detached and folded towards the posterior. Also the caudofemoralis was transected in order to completely expose the sciatic nerve [52].

A thermoregulatory device was customized to provide localized temperature modulation to the exposed rat sciatic nerve. The apparatus consisted of a flexible tube (Bev-A-Line IV, out diameter at 4.8 mm, inner diameter at 3.2 mm, thermoplastic processes, NJ, USA), which was inserted through 5 mm sub-muscular incisions and placed in parallel at about 5 mm to the left of the in situ sciatic nerve as shown in FIG. 4C.

### Thermoregulation

Temperature controlled water was continually circulated through the tube for immediate cooling and subsequent rewarming of the sciatic nerve using Blanketrol II system (Cincinnati Sub-zero, OH, USA), as shown in FIGS. 4A and 4B. A thermal blanket was placed on the ventral surface of the animal in order to maintain core body temperature at normothermia (37±0.5° C.). Thermocouple probes were used to monitor tympanic, rectal and sciatic nerve temperatures (prior to trifurcation) (as indicated in FIGS. 4A and 4C). The temperature data were recorded at 2 Hz using Thermes USB acquisition system and proprietary software (Physitemp, N.J., USA).

The temperature modulation protocol consisted of three stages: baseline, cooling and rewarming for all experimental animals (FIG. 5C). Baseline stage was recorded while the sciatic nerve temperature remained at 33±2° C. The cooling stage began 20 minutes after the onset of baseline recording. At this point, the sciatic nerve temperature was monotonically decreased by at least 10° C. at an approximate rate of −0.5° C./min. The cooling period ended after a plateau was maintained for 40 minutes. Subsequently, the sciatic nerve temperature was reverted to its baseline target during the rewarming stage, with a monotonic increase of about 0.5° C./min. The experiment concluded after 20 minutes of a maintained rewarmed state.

### Data Analysis of the Functional Changes in HbT and SO₂

Two optimized wavelengths (i.e., λ₅₆₀ and λ₅₇₀) were employed for monitoring the functional HbT and SO₂ changes with a high SNR and sensitivity [23]. The optical absorption of blood at λ₅₅₀ is sensitive to SO₂ levels, while the blood absorption at λ₅₇₀ results from the isobestic point of molar extinction spectra for oxy- and deoxy-hemoglobin [49, 53]. Because the λ₅₇₀ PA signal at a given pixel is proportional to the HbT within its resolution cell centered at that pixel, the mean functional HbT changes (R_{HbT} (t)) in the selected sciatic nerve region can be assessed as follows:

$${R}_{HbT} ( t ) = \frac{{\sum }_{( x , z ) ∈ ROI pixel} ( {I}_{( 570 )} ( x , z , t ) ) / A ( {I}_{( 570 )} ( {t}_{0} ) )}{{\sum }_{( x , z ) ∈ ROI pixel} ( {I}_{( 570 )} ( x , z , {t}_{0} ) ) / A ( {I}_{( 570 )} ( {t}_{0} ) )} , \qquad ( 1 )$$

where (x, z) is the pixel position; I₍₅₇₀₎(x, z, t) is the PA image at λ₅₇₀ acquired at time t and I₍₅₇₀₎ (x, z, t₀) is the baseline PA signal at λ₅₇₀ acquired immediately before the onset of cooling (i.e. at the baseline t₀); A(I₍₅₇₀₎(t₀)) represents the total pixel count of regions of interest (ROI) at the baseline t₀ [49]. Here, the ROI pixel was defined as the pixel that possessed a PA signal that was at least three times greater than the background signal [50, 54].

Functional images of SO₂ changes (I_{F(560)}(t)) at a given time point, t, at each stage were assessed according to the following equation:

$${I}_{F ( 560 )} ( t ) = \frac{{I}_{( 560 )} ( t )}{{I}_{( 570 )} ( t )} , \qquad ( 2 )$$

where I₍₅₆₀₎(t), i.e., PA image acquired at λ₅₆₀, was normalized to I₍₅₇₀₎(t) on a pixel-by-pixel basis [18]. The mean functional SO₂ changes (R_{SO}_{₂}(t)) in a single ROI region during the stimulation period were probed as follows:

$${R}_{{SO}_{2}} ( t ) = \frac{{\sum }_{( x , z ) ∈ ROI pixel} ( {I}_{F ( 560 )} ( x , z , t ) ) / A ( {I}_{( 570 )} ( {t}_{0} ) )}{{\sum }_{( x , z ) ∈ ROI pixel} ( {I}_{F ( 560 )} ( x , z , {t}_{0} ) ) / A ( {I}_{( 570 )} ( {t}_{0} ) )} \times 100 \% \qquad ( 3 )$$

That is, an independent probing of the changes in HbT and SO₂ could be achieved where I₍₅₇₀₎ was used as a marker for HbT, and I_{F(560)} was used-as a marker for SO₂ [49, 50, 55].

This experiment was designed to quantitatively compare the differences in relative PA signal changes from temperature modulation in the vasculature of the rat sciatic nerve. In the current fPAM setting, the data acquisition time for each PA B-scan image with 31 scanned lines (2 mm width) is about 28 seconds. Hence, it takes about 56 seconds for one functional image of I_{F(560)}. To identify vascular changes in response to temperature modulation, functional ultrasound and PA images were registered at a fixed cross-sectional area. Images acquired from the ultrasound scanning of the sciatic nerve were used as a reference to identify morphological characteristics and the PA region of interest (ROI) as indicated by the red dashed line in FIGS. 5B and 5C.

Statistical significance was assessed using a paired t-test with significance defined as p-value of <0.05 for the side-to-side differences in. PA signals (I_{R(570)} and I_{F(560)}) of the studied areas. The significance of changes observed in fPAM signals (I_{F(560)}) at the respective ROI in response to temperature modulation was compared using the Wilcoxon matched-pairs signed-rank test (two-tailed, p<0.05, n=10) [50, 51, 55]. All statistical analyses were performed using SPSS (version 10.0, SPSS®, USA).

### EXAMPLE 1

An embodiment of the invention is shown in FIG. 1 which mainly consists of (1) a cooling means for example a cooling controller; and (2) the device according to the invention. The cooling controller constantly pumps the coolant (for example chilled distilled water) to the limb coverings to induce local cooling to the limbs. It can receive feedback signals via the temperature sensors to control the temperature of coolant for maximal peripheral nerve protection during chemotherapy. The devices according to the invention can be designed to comfortably fit to patient's arms and legs and can be taken off easily. The device can be provided with a layer of fabric in between the skin and device for better heat comfort for the patient (FIGS. 2a and 2b). The objective is to ensure high coolant velocity throughout the flow field covering the entire device so as to maintain uniform cooling to the device when in use.

### EXAMPLE 2

Photoplethysmography (PPG) is a simple and low-cost optical technique that can be used to detect blood volume changes in the micro-vascular bed of tissue. It is often used non-invasively to make measurements at the skin surface. PPG is a non-invasive optical technique for detecting microvascular blood volume changes in tissues due to the pulsatile nature of the circulatory system. PPG technology (reflection mode) for imaging blood perfusion is shown in FIG. 3a. It requires a trans-illuminated cooling device, which not only effects cooling but also allows incident light and reflective light to travel through. FIGS. 4b and 4c show the strong correlation between temperature and PPG signals. PPG technology for imaging blood perfusion during induced hyperthermia and chemotherapy using the device according to the invention is an important biomarker to monitor hypothermia in a patient. FIG. 3b illustrates a sample of layered PPG imaging of blood perfusion in the palm. The color indicates the intensity (a.u.). The depth of optical penetration (and hence the measurement depth) can be controlled by choosing light sources with different wavelengths. This allows measurement of blood perfusion for various nerve endings in different layers of skin's inner tissue. PPG (reflection mode) can be used to monitor tissue oxygenation (SpO₂). It utilizes PPG measurements to obtain information about oxygen saturation by shining red and then near infrared light through vascular tissue, with rapid switching between the wavelengths. The amplitudes of the red and near infrared AC signals are sensitive to changes in oxygen saturation because of differences in the light absorption of HbO₂ and Hb at these two wavelengths. From their amplitude ratio, and corresponding PPG DC components, SpO₂ can be estimated.

### EXAMPLE 3

### PA Imaging of the Rat Sciatic Nerve Vasculature

Dark-field functional photoacoustic microscopy (fPAM) is a suitable method for intrinsic visualizing of the relative hemodynamics of the rat sciatic nerve in response to localized temperature modulation (i.e., cooling and rewarming).

A photograph of the surface of rat sciatic nerve is shown in FIG. 5A. Many distinct blood vessels varying in size can be seen at the epineurium of the sciatic nerve. The B-scan ultrasound and photoacoustic (PA) images of the rat sciatic nerve are shown in FIGS. 5B and 5C, respectively. Increased relative HbT and SO2 values peak in the same areas that the blood vessel are seen, suggesting that these regions represent blood vessels as visualized by fPAM (FIG. 5C).

### EXAMPLE 4

### Localized Thermoregulation Response

A selected cross-section region of the sciatic nerve was examined for relative side-to-side PA signal differences in response to localized temperature modulation. The thermoregulation protocol was designed to quantitatively compare the relative HbT and SO₂ changes in vascular structures of the sciatic nerve between three localized temperature modulation stages: baseline, cooling and rewarming (FIG. 5D). FIG. 6A shows the response for both relative HbT and SO₂ changes as function of time under the thermoregulation protocol.

### EXAMPLE 5

### Functional Temperature Dependent Hemodynamics in Sciatic Nerve

In vivo functional hemodynamics of the sciatic nerve in response to thermoregulation at difference stages are shown in FIG. 6A. The ultrasound and PA images are shown in FIGS. 5B and 5C, respectively, and their Regions of Interest (ROIs) were used for the statistical analysis. Both relative HbT and SO₂ changes demonstrate significant correlations with localized thermoregulation during cooling and rewarming stages, as shown in FIGS. 6B and 6C, respectively (p<0.05; paired t-test). Quantitative analysis shows that during cooling, relative HbT (R_{HbT}) was reduced by −70.8% with respect to its baseline, and correlated with temperature by r=0.94. Similarly, relative SO2 (R_{SO2}) was lowered by −73.3% from its baseline value, and correlated with temperature by r=0.88. The applied thermoregulation protocol did not significantly affect R_{HbT} and [symbol] R_{SO2} in being restored to its initial baseline values (p >0.05; paired t-test).

### REFERENCES

1. Stubblefield, M. D., et al., NCCN task force report: management of neuropathy in cancer. J Natl Compr Canc Netw, 2009. 7 Suppl 5: p. S1-S26; quiz S27-8.
2. Gilchrist, L., Chemotherapy-induced peripheral neuropathy in pediatric cancer patients. Semin Pediatr Neurol, 2012. 19(1): p. 9-17.
3. Windebank, A. J. and W. Grisold, Chemotherapy-induced neuropathy. J Peripher Nery Syst, 2008. 13(1): p. 27-46.
4. Ghoreishi, Z., et al., Omega-3 fatty acids are protective against paclitaxel-induced peripheral neuropathy: A randomized double-blind placebo controlled trial. BMC Cancer, 2012. 12(1): p. 355.
5. Tanabe, Y., et al., Paclitaxel-induced peripheral neuropathy in patients receiving adjuvant chemotherapy for breast cancer. Int J Clin Oncol, 2011.
6. Howlader, N., et al., SEER Cancer Statistics Review 1975-2009 (Vintage 2009 Populations). Bethesda, Md.: National Cancer Institute. Retrieved June, 2012. 18: p. 2012.
Peng, L. H., et al., Singapore Cancer Registry Interim Annual Registry Report Trends in Cancer Incidence in Singapore 2006-2010, 2006-2010, National Registry of Diseases Office (NRDO): Singapore.
8. Fitch, K. and B. Pyenson, Cancer Patients Receiving Chemotherapy: Opportunities for Better Management, in Milliman Client Report, I. Milliman, Editor 2010, Innovent Oncology: NY.
9. Dworkin, R. H., et al., Impact of postherpetic neuralgia and painful diabetic peripheral neuropathy on health care costs. Journal of Pain, 2010. 11(4): p. 360.
10. Polderman, K. H., Induced hypothermia and fever control for prevention and treatment of neurological injuries. Lancet, 2008. 371(9628): p. 1955-69.
11. Sun, X., W. Tang, and L. Zheng, Ultrastructural observation of effect of moderate hypothermia on axonal damage in an animal model of diffuse axonal injury. Chin J Traumatol, 2002. 5(6): p. 355-60.
12. Tsai, Y. J., et al., Effects of regional and whole-body hypothermic treatment before and after median nerve injury on neuropathic pain and glial activation in rat cuneate nucleus. Anesthesiology, 2012. 116(2): p. 415-31.
13. Zhao, H., G. K. Steinberg, and R. M. Sapolsky, General versus specific actions of mild-moderate hypothermia in attenuating cerebral ischemic damage. J Cereb Blood Flow Metab, 2007. 27(12): p. 1879-94.
14. Badjatia, N., Hyperthermia and fever control in brain injury. Crit Care Med, 2009. 37(7 Suppl): p. S250-7.
15. Uray, T. and R. Malzer, Out-of-hospital surface cooling to induce mild hypothermia in human cardiac arrest: a feasibility trial. Resuscitation, 2008. 77(3): p. 331-8.
16. Li, J., S. Benashski, and L. D. McCullough, Post-stroke hypothermia provides neuroprotection through inhibition of AMP-activated protein kinase. J Neurotrauma, 2011. 28(7): p. 1281-8.
17. Bayegan, K., et al., Rapid non-invasive external cooling to induce mild therapeutic hypothermia in adult human-sized swine. Resuscitation, 2008. 76(2): p. 291-8.
18. Budnick, B., K. L. McKeown, and W. C. Wiederholt, Hypothermia-induced changes in rat short latency somatosensory evoked potentials. Electroencephalogr Clin Neurophysiol, 1981. 51(1): p. 19-31.
19. Kelly, C., et al., Regional hypothermia protects against tourniquet neuropathy. Eur J Vasc Surg, 1992. 6(3): p. 288-92.
20. Mitsui, Y., et al., Hypothermic neuroprotection of peripheral nerve of rats from ischaemia-reperfusion injury. Brain, 1999. 122 (Pt 1): p. 161-9.
21. Stecker, M., J. Wolfe, and M. Stevenson, Neurophysiologic responses of peripheral nerve to anoxia: Effects of hypothermia and age. Clin Neurophysiol, 2012.
22. Stecker, M. M. and K. Baylor, Peripheral nerve at extreme low temperatures 1: effects of temperature on the action potential. Cryobiology, 2009. 59(1): p. 1-11.
23. Massey, C. S., A multicentre study to determine the efficacy and patient acceptability of the Paxman. Scalp Cooler to prevent hair loss in patients receiving chemotherapy. Eur J Oncol Nurs, 2004. 8(2): p. 121-30.
24. van den Hurk, C. J., et al., Scalp cooling for hair preservation and associated characteristics in 1411 chemotherapy patients—results of the Dutch Scalp Cooling Registry. Acta Oncol, 2012. 51(4): p. 497-504.
25. Pennes, H. H., Analysis of tissue and arterial blood temperatures in resting human forearm. Journal of Applied Physiology, 1948. 1: p. 93-122.
26. Hertzman, A. and C. Spealman, Observations on the finger volume pulse recorded photoelectrically. Am. J. Physiol, 1937. 119(334): p. 3.
Hertzman, A. B., The blood supply of various skin areas as estimated by the photoelectric plethysmograph. Am. J. Physiol., 1938. 124(2): p. 328-340.
28. Allen, J., Photoplethysmography and its application in clinical physiological measurement. Physiol. Meas., 2007. 28(3): p. R1-39.
29. Suichies, H. E., et al., Digital blood flow in cooled and contralateral finger in patients with Raynaud's phenomenon. Comparative measurements between photoelectrical plethysmography and laser Doppler flowmetry. Angiology, 1992. 43(2): p. 134-41.
30. Rosato, E., et al., The combination of laser Doppler perfusion imaging and photoplethysmography is useful in the characterization of scleroderma and primary Raynaud's phenomenon. Scand. J. Rheumatol., 2011. 40(4): p. 292-8.
31. Allen, J., J. R. Frame, and A. Murray, Microvascular blood flow and skin temperature changes in the fingers following a deep nspiratory gasp. Physiol. Meas., 2002. 23(2): p. 365-73.
32. Raamat, R., et al., Continuous mean arterial pressure measurement in the fingers: the influence of local arm cooling. Med. Biol. Eng. Comput., 2001. 39(5): p. 584-9.
33. Kyriacou, P. A., Pulse oximetry in the oesophagus. Physiol Meas, 2006. 27(1): p. R1-35.
34. Yu, J., et al. Prefrontal Cortical Activation during Arithmetic Processing Differentiated by Cultures: A Preliminary fNIRS Study. in Proceedings of the 34th Annual International Conference of the IEEE Engineering in Medicine and Biology Society. 2012. San Diego, Calif., USA.
35. Ang, K. K., J. Yu, and C. Guan. Extracting effective features from high density NIRS-based BCI for assessing numerical cognition. in IEEE International Conference on Acoustics, Speech and Signal Processing. 2012.
36. Ang, et al. A Brain-Computer Interface for Mental Arithmetic Task from Single-Trial Near-Infrared Spectroscopy Brain Signals. in 20th International Conference on Pattern Recognition. 2010.
37. Sitaram, R., et al., Temporal classification of multichannel near-infrared spectroscopy signals of motor imagery for developing a brain-computer interface. Neurolmage, 2007. 34(4): p. 1416-1427.
38. Stein, E. W., K. Maslov, and L. V. Wang, Noninvasive, in vivo imaging of blood-oxygenation dynamics within the mouse brain using photoacoustic microscopy. Journal of Biomedical Optics, 2009. 14(2): p. 020502.
39. Zhang, H. F., et al., Functional photoacoustic microscopy for high-resolution and noninvasive in vivo imaging. Nature Biotechnology, 2006. 24: p. 848-851.
40. Wang, L. V., Multiscale photoacoustic microscopy and computed tomography. Nature Photonics, 2009. 3: p. 503-509.
41. Wang, L. V., Tutorial on photoacoustic microscopy and computed tomography. IEEE Journal of Selected Topics in Quantum Electronics, 2008. 14(1): p. 171-179.
42. Fainchtein, R., et al., Local determination of hemoglobin concentration and degree of oxygenation in tissue by pulsed photoacoustic spectroscopy. Proceedings of SPIE, 2000. 3916: p. 19-33.
43. Maslov, K., G. Stoica, and L. V. Wang, In vivo dark-field reflection-mode photoacoustic microscopy. Optics Letter, 2005. 30(6): p. 625-627.
44. Stein, E. W., K. Maslov, and L. V. Wang, Noninvasive mapping of the electrically stimulated mouse brain using photoacoustic microscopy. Proceedings of SPIE, 2008. 6856: p. 685654.
45. Schag, C. C., R. L. Heinrich, and P. A. Ganz, Karnofsky performance status revisited: reliability, validity, and guidelines. J Clin Oncol, 1984. 2(3): p. 187-93.
46. Cavaletti, G., et al., The Total Neuropathy Score as an assessment tool for grading the course of chemotherapy-induced peripheral neurotoxicity: comparison with the National Cancer Institute-Common Toxicity Scale. J Peripher Nery Syst, 2007. 12(3): p. 210-5.
47. Cavaletti, G., et al., The chemotherapy-induced peripheral neuropathy outcome measures standardization study: from consensus to the first validity and reliability findings. Ann Oncol, 2012.
48. Bennett, M., The LANSS Pain Scale: the Leeds assessment of neuropathic symptoms and signs. Pain, 2001. 92(1-2): p. 147-57.
49. L. D. Liao, V. Tsytsarev, I. Delgado-Martinez, M. L. Li, R. Erzurumlu, A. Vipin, J. Orellana, Y. R. Lin, H. Y. Lai, Y. Y. Chen, and N. V. Thakor, “Neurovascular coupling: in vivo optical techniques for functional brain imaging,” Biomedical Engineering Online, vol. 12, p. 38, Apr. 30, 2013.
L.-D. Liao, M.-L. Li, H.-Y. Lai, Y.-Y. I. Shih, Y.-C. Lo, S. Tsang, P. C.-P. Chao, C.-T. Lin, F.-S. Jaw, and Y.-Y. Chen, “Imaging brain hemodynamic changes during rat forepaw electrical stimulation using functional photoacoustic microscopy,” NeuroImage, vol. 52, pp. 562-570; 2010.
51 A. Rupp, U. Dornseifer, A. Fischer, W. Schmahl, K. Rodenacker, U. Jutting, P. Gals, E. Biemer, N. PapadopUlos, and K. Matiasek, “Electrophysiologic assessment of sciatic nerve regeneration in the rat: surrounding limb muscles feature strongly in recordings from the gastrocnemius muscle,” J Neurosci Methods, vol. 166, pp. 266-77, Nov. 30, 2007.
52 Stein E W, Maslov K, Wang L V: Noninvasive, in vivo imaging of blood-oxygenation dynamics within the mouse brain using photoacoustic microscopy. J Biomed Opt 2009, 14:020502
53 L. Li, R. J. Zemp, G. Lungu, G. Stoica, and L. V. Wang, “Photoacoustic imaging of IacZ gene expression in vivo,” Journal of Biomedical Optics, vol. 12, p. 020504, 2007.
54 L. D. Liao, C. T. Lin, Y. Y. I. Shih, T. Q. Duong, H. Y. Lai, P. H. Wang, R. Wu, S. Tsang, J. Y. Chang, and M. L. Li, “Transcranial imaging of functional cerebral hemodynamic changes in single blood vessels using in vivo, photoacoustic microscopy,” J Cereb Blood Flow Metab, vol. 32, pp. 938-951, 2012.
55 Liao L-D, Lin C-T: Novel trends in biosensors used for electroencephalography measurements in neurocognitive engineering applications. J Neurosci Neuroeng 2012, 1:1006.
56 H. F. Zhang, K. Maslov, and L. V. Wang, “In vivo imaging of subcutaneous structures using functional photoacoustic microscopy,” Nat Protoc, vol. 2, pp. 797-804, 2007.

1. A device for inducing regulated controlled hypothermia in a subject comprising: a body part covering comprising flexible material adapted to contact the skin of a subject and having an upper and lower surface defining a space for coolant, the device provided with one or more inlets for fluid coolant connecting to a closed network of channels to facilitate even distribution of fluid within the device space and describing a coolant dispersal pattern when in use to provide uniform cooling to the subjects body part and wherein the device is further provided with one or more temperature sensors and one or more blood flow sensors to monitor the subjects body part temperature and/or the blood flow through the subjects body part.

2. The device according to claim 1, wherein the device is adapted to cover at least partially the upper limb[s] of the subject and/or cover at least partially the lower limb[s] of the subject.

3. The device according to claim 2 wherein said device is adapted to cover at least the hands and forearm of the subject.

4. (canceled)

5. The device of claim 1, wherein said device is provided with:
at least one temperature sensor and at least one blood flow sensor in functional contact with the hand, forearm and/or lower limb of the subject; or
a plurality of temperature sensors and a plurality of blood flow sensors in functional contact with the hand, forearm and/or lower limb at the subject.

6. (canceled)

7. The device according to claim 1, wherein said device is provided with multiple inlets.

8. The device of claim 1, wherein the channels are formed from a plurality of upwardly projecting fins which are positioned near to each other with the cavity of the body part covering to create a network of channels throughout the body part covering.

9. The device according to claim 8 wherein the fins are positioned equidistant from each other within the cavity of the body part covering.

10. The device of claim 1, wherein said body covering is provided with more than one sensor functionally linked in series to provide continuous temperature and/or blood flow monitoring of said subject over all or part of the subjects body part and adjusting the cooling means to control the induced hypothermia.

11. The device of claim 1, wherein said device further comprises a plurality of micro temperature probes adapted to measure the skin temperature of the subject's body part when in use and wherein the micro temperature probes are positioned to provide a measure of the skin temperature over the area of the patient's body contacted by the device.

12. The device of claim 1, wherein said flexible material manufactured from a material transparent to light or is optically clear.

13. (canceled)

14. The device of claim 1, wherein said blood flow sensor[s] detect the concentration of haemoglobin and/or the oxygenation of haemoglobin.

15. (canceled)

16. An apparatus for inducing and regulating controlled hypothermia in a subject to prevent or treat peripheral nerve neuropathy, comprising:
the device of claim 1, wherein the device is functionally connected to a temperature and/or blood flow monitor and/or oxygenation monitor to monitor temperature and/or blood flow and/or oxygenation in the subject's body part via the temperature and/or blood flow sensors;
a cooling means which when is use delivers coolant to the body covering which induces hypothermia in the subject and is regulated when in use to provide optimal cooling to the subject; and
optionally a photoacoustic microscopy system for the monitoring of blood flow.

17. (canceled)

18. A method to monitor nerve temperature during chemotherapy induced hypothermia in a subject, comprising:
i) providing the device of claim 1, wherein the device includes a plurality of micro temperature probes and connecting the device to said subject; and
ii) monitoring the skin temperature of the subject during induced hypothermia.

19. A method for continuous monitoring of blood flow and/or blood oxygenation through a subject's body part during induced hypothermia and chemotherapy, comprising:
i) applying the device of claim 1 to a subject in need of chemotherapy, wherein the device is manufactured from transparent material;
ii) exposing the device to a light source; and
iii) measuring the flow of blood and/or oxygenation through and of the body part.

20.-21. (canceled)

22. The method according to claim 19 wherein the concentration of haemoglobin is monitored as a measure of blood flow and/or wherein the oxygenation of haemoglobin is monitored as a measure of blood flow.

23.-26. (canceled)

27. A method to prevent or treat peripheral nerve neuropathy during chemotherapy, comprising:
i) connecting the device of claim 1 to a subject administered or about to be administered a chemotherapeutic agent;
ii) cooling the subject to induce hypothermia in one or more parts of the subject's body; and
iii) monitoring and controlling the temperature of the subject to maintain induced hypothermia.

28. The method according to claim 27 wherein the temperature of the subject's body part is reduced to between 15° C. and 25° C.

29.-30. (canceled)

31. The method according to claim 27, wherein the induced hypothermia is maintained:
for a period after administration of chemotherapy;
for a period before administration of chemotherapy;
for a period of 3 hours after administration of chemotherapy;
throughout the administration of chemotherapy;
for a period of about 30 minutes after administration of chemotherapy;
for a period of about 30 minutes before administration of chemotherapy;
for a period up to 3 hours after administration of chemotherapy; or
for a period up to 3 hours before administration of chemotherapy.

32.-36. (canceled)

37. The method of claim 27, wherein said subject is human.

38. The method of claim 27, wherein the peripheral nerve neuropathy is:
chemotherapy induced neuropathy as a result of cancer treatment; or
infection induced.

39. The method according to claim 38 wherein said cancer treatment is breast cancer treatment.

40. The method of claim 38, wherein the chemotherapy induced neuropathy is the result of administration of a chemotherapeutic agent selected from the group consisting of: vincristine, vinca alkaloids, platinum compounds such as cisplatin, oxaliplatin, carboplatin, taxanes, epothilones, bortezomib and thalidomide.

41. (canceled)

42. The method according to claim 38, wherein said infection is an HIV infection.

43. The method according to claim 42 wherein peripheral neuropathy as a consequence of HIV infection is due to direct viral infection.

44. The method according to claim 42, wherein peripheral neuropathy as a consequence of HIV infection is as a consequence of administration of anti-viral chemotherapy.

45. The method of claim 27, wherein peripheral nerve neuropathy is associated with an inflammatory neural disease.

46.-47. (canceled)

48. The method of claim 27, wherein peripheral nerve neuropathy is associated with an endocrine or metabolic disease.

49. (canceled)

50. The method of claim 27, wherein:
chemotherapy treatment is administered before, during or after induction of hypothermia;
said subject is precooled prior to administration of a chemotherapeutic agent; and/or
induced hypothermia is combined with the administration of neuroprotective agents.

51.-54. (canceled)
